(19) United States
(12) Patent Application Publication
(10) Pub. No.: US 2013/0052978 A1
(43) Pub. Date: Feb. 28, 2013

(54) Seizure Assist Dog Method and Equipment

(71) Applicant: Jon C. Sabin, South Colton, NY (US)

(72) Inventor: Jon C. Sabin, South Colton, NY (US)

(21) Appl. No.: 13/221,226

(22) Filed: Aug. 30, 2011

(51) Int. Cl. A61N 1/372; H04W 4/22

(52) U.S. Cl. 455/404.1; 607/45

(57) ABSTRACT

A system for controlling epileptic seizure episodes is provided for a patient who is accompanied by a service dog trained to watch the patient and respond to seizure episodes. The patient is provided with a VNS generator implanted into his or her chest, with electrodes or lead that to the patient's vagus nerve in the neck. The service dog's collar is fitted with a VNS permanent magnet device. The dog, being trained to sense the onset of a seizure episode in said patient, will move onto the patient so that so that the dog collar comes into the zone of the generator on the patient's chest. This causes the generator to send prescribed stimulation to the vagus nerve. The dog is also trained to bite down on a cell phone case containing a phone carried by the patient or by the dog, which then automatically phones a preset number to summon human assistance.

### BACKGROUND OF THE INVENTION

The present invention concerns therapies for prevention and treatment of seizures in epilepsy patients. The invention is more particularly concerned with the use of VNS stimulation to modify the effects of epilepsy, and is also directed to the use of a service dog trained to respond to the onset of an epileptic seizure in a patient that the dog is trained to accompany.

The practice of treating an epilepsy patient with a vagus nerve stimulation or VNS is well known. The VNS device is widely used as a therapeutic means to control epileptic seizures or at least to reduce the incidence of seizures in the patient. The VNS device or generator is typically an implant device, and requires surgical implantation, usually as an outpatient procedure.

A VNS device is usually contained in a titanium case about two inches in diameter, with electrodes or leads that leave the case and attach to the patient's vagus nerve. These devices have a battery life that can exceed a decade. The VNS generator is inserted into the patient's upper left chest via a small incision. Another incision is made in the neck to access the patient's left vagus nerve. The surgeon wraps the lead or leads around the vagus nerve and connects the electrodes to the generator. Once implanted, the generator sends electric impulses to the vagus nerve at regular intervals. The strength, timing, and pattern of these pulses are determined by the patient's neurologist, and the generator is programmed accordingly. VNS therapy has proven to be generally effective for treating partial onset epilepsy.

The generator also includes a magnetic switch or sensor, such that if a strong magnet is swiped past the implanted device, it will generate stimulative impulses of a different strength and/or pattern, which are then carried over the electrodes to the patient's vagus nerve. The patient is provided with a magnetic bracelet so that the patient can swipe the bracelet through a zone in the left chest near the implant when the patient feels that an epileptic seizure is or may be coming on. While this additional stimulation is often effective in forestalling a full seizure, it is often the case that the patient is physically unable to carry out a swipe maneuver due to changes at the beginning of the seizure.

Recently, service dogs have been trained for epilepsy patients and assigned to accompany and monitor them, i.e, watch them. These dogs are trained to react when the patient shows signs of the beginning of an epileptic fit. However, to date the dogs have been trained only to protect the patient during the seizure, for example by pulling off blankets or coverings to prevent the patient from asphyxiation, or to lie upon the patient to prevent involuntary movement of the arms and legs from causing injury to the patient. To date, no one except the inventor herein has linked seizure dogs, i.e., service dogs trained for epilepsy patients, to the actuation of any sort of therapeutic or electronic device to help forestall or prevent a seizure. No one else has provided a simple system for a service dog to cause a phone to dial an emergency number to summon human assistance when the patient suffers an epileptic seizure.

### SUMMARY OF THE INVENTION

Accordingly, it is an object of this invention to employ a trained service dog to assist an epilepsy patient in application of VNS therapy, as needed. It is also an object to provide a means for the service dog to summons human assistance when the patient that it is accompanying suffers an epileptic seizure using a common cellular phone.

A preferred system is provided for controlling seizure episodes in a patient who suffers from epilepsy and who is accompanied by a service dog trained to watch the patient and respond to seizure episodes. A vagus nerve stimulation implant device, e.g., a VNS generator, is fitted into the chest of he patient, the device having one or more leads or electrodes coupled to the vagus nerve of the patient. The VNS generator is programmed to generate therapeutic stimulation and to transmit this along the electrode to the patient's vagus nerve in a pattern to assist in control for epilepsy. In this case, the implant device includes a magnetic actuator or sensor that causes the device to generate a stimulative signal to pass, on lead or electrode to the patient's vagus nerve when it senses a magnetic field from an external magnet device being swiped across a zone on the patient's chest near the implant device. The service dog is specially trained to sense the onset of a seizure episode in the patient and is provided with a dog collar worn on its neck, and the collar has a permanent magnet device affixed onto it. When the service dog senses the onset of a seizure episode, the dog maneuvers onto the left shoulder of the patient so that the dog collar comes into the aforesaid zone on the patient's chest. This action swipes the magnet through the zone so that the implant, i.e., the VNS generator, provides the stimulation to the vagus nerve that in many or most cases will prevent or at least abbreviate the seizure. In a preferred embodiment, there is a strap attaching the external permanent magnet device to the dog collar. A pair of fasteners each include a female threaded ferrule set into the dog collar and a retaining screw threaded to fit the associated ferrule. This permits the device to be easily installed or removed on the dog collar.

Here, the service dog is a “seizure dog,” and has been trained use its keen sense of smell to changes in the patient's body chemistry associated with the onset of a seizure episode, which are changes undetectable to the human sense of smell. The dog is trained to respond to these and to execute the above-mentioned maneuver, as well as to take other actions for which it is trained to protect the seizure victim. The service dog is also trained to watch the patient's facial expression and to respond to very subtle changes in facial expression that may be characteristic of the onset of a seizure episode.

According to another aspect of the invention, the seizure prevention and control system can also employ a cellular portable phone, of the type that has with a dial pad that includes at least one button that can be set to cause the phone to dial an emergency contact number when the button is depressed. This can be a physical button or a touch screen button. The cell phone is fitted into a special case that is formed of a stiff material. The case possesses a protuberance, e.g., a dome or ball, inside the case that is positioned over the aforesaid button of the cell phone. The service dog is trained to grab the cell phone with his mouth when the dog senses that the patient is at the onset of a seizure episode, and then to clamp down and chew on the case. This serves to depress the protuberance in the case into the button, which then causes the cell phone to dial the emergency contact number. This can be the number of an emergency service, or the number of a family member or other person who would expect to respond when phoned in this way. The cell phone case is attached by a strap or lanyard either to be suspended from the patient (i.e., hanging from the patient's belt) or from the service dog itself. This places the case with the cell phone in position so that it can be readily grasped by the service dog at the onset of a patient's seizure episode.

The service dog is trained, as mentioned before, to identify, with its sense of smell, changes in the body chemistry of the patient that characterize the onset of a seizure episode, and to identify changes in the person's aspect, i.e., changes in the facial expression, which also may indicate the onset of a seizure episode.

In a favorable embodiment, the case has a leather outer shell and a stiff liner. The protuberance can be in the form of a round-head rivet affixed in the case at the position of the button that dials the emergency phone number.

The cell phone may also be programmed for normal phone use, and to return to emergency use when replaced into the case.

One particular preferred embodiment is illustrated in the accompanying Drawing figures, which illustrate one implementation of the main principles of the invention.

### DESCRIPTION OF THE DRAWING

FIG. 1 is a schematic view of an embodiment of the system of my invention, showing an epilepsy patient with an implanted VNS generator and a suitably equipped and trained service dog.

FIG. 2 is a perspective view of the service dog showing the dog collar with the permanent magnet VNS actuator affixed thereon.

FIG. 3 is a view showing the dog collar, a choke collar that can be used for suspending the cell phone and case, a belt lanyard and the cell phone case, as employed in the system of one embodiment of my invention.

FIG. 4 is a front view of the cell phone and the cell phone case.

FIG. 5 is a rear view of the cell phone case, with the associated phone being inserted into it.

FIG. 6 is a plan view of the dog collar with the permanent magnet actuator affixed thereon as used in this embodiment.

FIGS. 7 and 8 are enlarged cross sections for explaining the construction of the dog collar of FIG. 6.

### DETAILED DESCRIPTION OF A PREFERRED EMBODIMENT

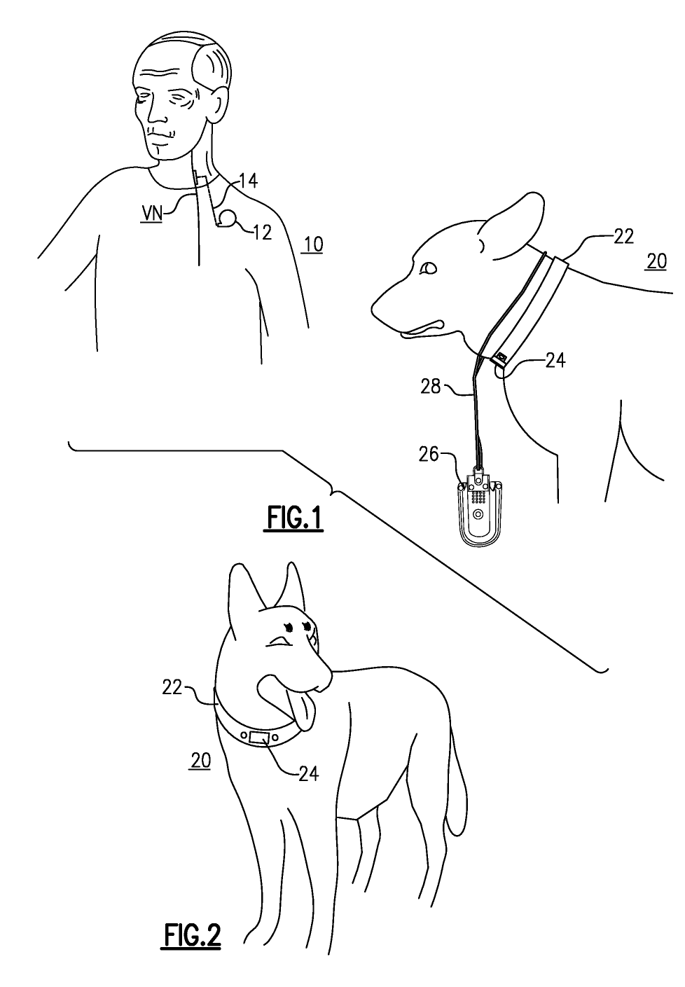

As shown in FIGS. 1 and 2, a human epilepsy patient 10 is provided with a vagus nerve stimulation generator 12 surgically implanted in his chest. The generator has one or more electrodes or leads 14 that are coupled to the patient's left vagus nerve VN, within the patient's neck. The implant of the generator 12 and the connection of the electrodes 14 to the patient's vagus nerve VN are carried out as an outpatient procedure, using small incisions in the chest and neck. For reasons having to do with the role of the right vagus nerve in controlling heart function, the generator 12 and leads 14 are connected only to the left vagus nerve. A service dog 20, here a German shepherd, is specially trained to accompany the patient 10, and is also trained to recognize signs that the patient is or may be in the onset stages of an epileptic seizure. The dog 20 is also trained to respond to the signs of an onset of a seizure event and to take some specific actions related to stimulating the VNS generator 12 and also to alert a human assistant via telephone. Here, the dog 20 is wearing a dog collar 22 that has a special permanent magnet device 24 affixed onto it and is also has a cell phone, contained in a special case 26, which is suspended from the dog's neck, in this instance using a rope choke collar 28 as a lanyard. Alternatively, the cell phone and case 26 can be suspended from the patient 10, e.g., using a strap or lanyard 30 that the patient may wear on his belt.

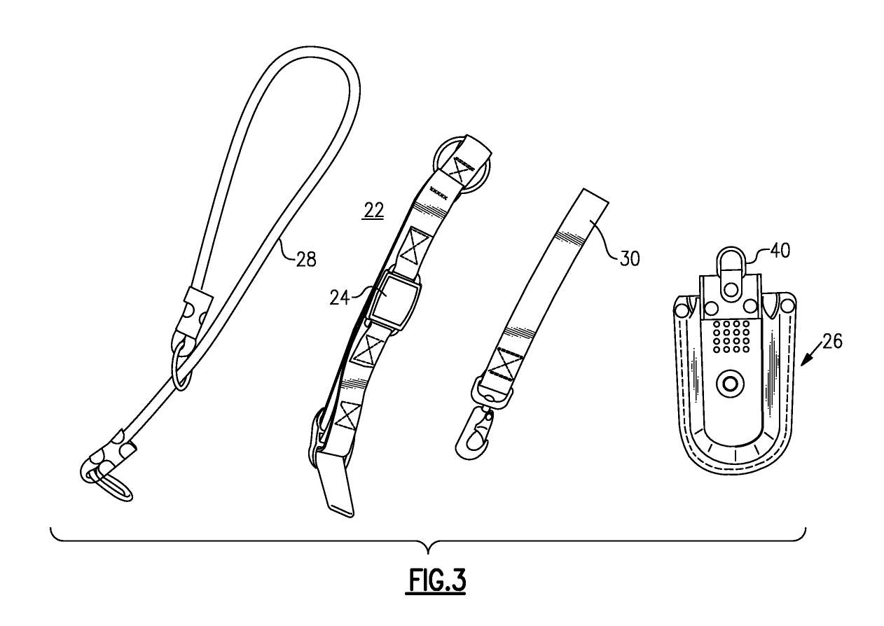

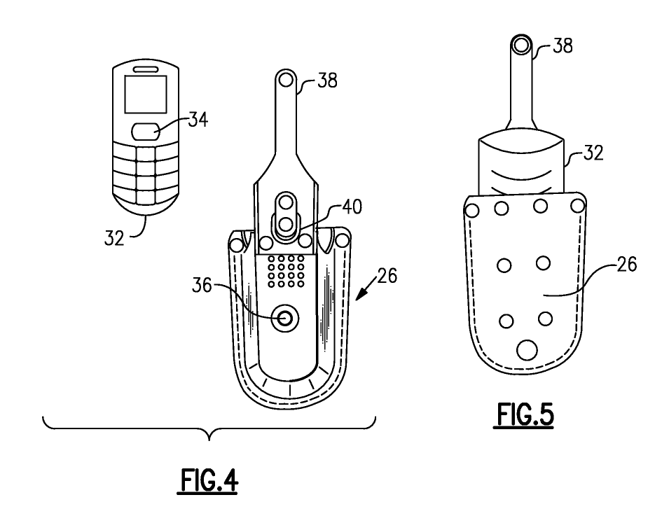

FIG. 3 shows an example of the choke collar 28, the dog collar 22 with the VNS actuator magnet device 24 mounted onto it; a lanyard 30 from which the patient 10 may suspend the cell phone case 26; and an example of the cell phone case 26, here shown closed and from the forward or front side. FIGS. 4 and 5 show a cellular phone 32 that is inserted into the case 26. The cell phone 32 has a dial button 34, and the phone is programmed to dial a predetermined phone number when the button 34 is depressed. The case 26 has a round-head rivet 36 mounted onto the front side of the case 26, with the head directed inward, and located at the position of the dial button 34. The case here is made of leather but has a stiff plastic liner, except at the location of the rivet 36. A closure strap 38 serves to secure the phone 32 in the case 26 when it is being used by the patient, and carried by the patient or the service dog. As shown in FIG. 5, the strap 38 can be secured through a pair of loops on the reverse side of the case, and then held down with a snap closure. Other equivalent fastening means could also serve this purpose. A ring 40 on the cell phone case 26 allows the phone to be suspended from either the patient or the dog, e.g., using the lanyard 30 or the choke collar 28.

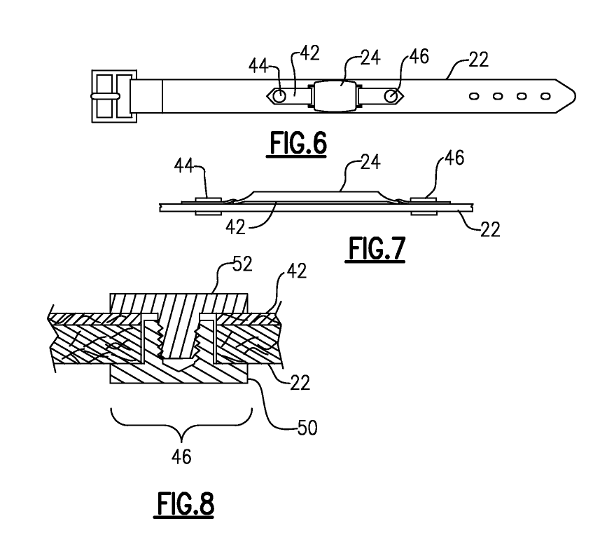

Details of the dog collar 22 employed in this embodiment are shown in FIGS. 6, 7 and 8. A magnet strap 42 is attached to the collar and can be affixed at one end to the collar 22 using a rivet 44, such as a pop rivet, and is secured at its other end to the collar using a screw rivet 46, as shown in more detail in FIGS. 7 and 8. The magnet strap passes through watch-band hold down ears 48 that are provided on opposite ends of the VNS magnet device 24. The screw rivet, shown in more detail in FIG. 8, has a female-threaded grommet 50 that is affixed in the collar 22 and a male threaded hold down 52 that passes through the magnet strap 42 and holds it to the grommet 50. This arrangement allows the magnet device 24 to be easily attached to the dog collar 22 and allows it to be removed and changed from one dog collar to another.

With this arrangement, the magnet device 24 is in the proper position so when the service dog 20 reaches up to the left side of the patient 10, as the dog has been trained to do when it senses the onset of a seizure episode, the magnet 24 will stimulate the VNS generator 12, and in many or most cases end the seizure or at least reduce its effect on the patient. If the seizure continues, the dog is trained to bite the cell phone case 26, and this causes the cell phone 32 to call a pre-set emergency phone number, which can be a care giver, a family member, a friend, or another person who will respond.

Many variations of this are possible, depending on the arrangement of the keypad on the phone, and depending on the specific training used for the service dog. While the invention has been described in terms of a preferred embodiment, it should be appreciated that many variations thereof are possible and would present themselves to persons of skill in the art without departing from the scope and spirit of this invention.

1. A system for controlling seizure episodes in a patient who suffers from epilepsy and who is accompanied by a service dog trained to watch the patient and respond to seizure episodes, the system comprising:
a vagus nerve stimulation implant device fitted into the chest of the patient, the device having an electrode coupled to the vagus nerve of the patient, and adapted to generate therapeutic stimulation to transmit along the electrode to the patient's vagus nerve in a pattern to assist in control of epilepsy, the implant device including a magnetic actuator to cause the device to generate a stimulative signal to pass on said electrode to said vagus nerve in response to a magnetic field from an external magnet device coming into a zone on the patient's chest near the implant device;
a dog collar to be worn by the service dog, the dog being trained to sense the onset of a seizure episode in said patient, and to contact the patient so that the dog collar comes into said zone on the patient's chest, said dog collar including said external magnet device affixed onto it;
a cell phone with a dial pad that includes at least one button that can be set to cause the phone to dial an emergency contact number when the button is depressed;
a case into which the cell phone is fitted, the case being formed of a stiff material, the case possessing a protuberance inside the case that is positioned over said button, and which serves to depress said button and cause the cell phone to dial said emergency contact number in the event that the service dog grasps the cell phone and case in its mouth and begins to chew; and
a strap that is attached to one of the patient or the service dog and to the case of the cell phone, so that the latter is in position to be grasped by the service dog at the onset of a seizure episode for said patient.

2. The system for controlling seizure episodes according to claim 1, further comprising a strap attaching said external magnet device to said dog collar, and a pair of fasteners each including a female threaded ferrule set into the dog collar and a retaining screw threaded to fit the associated ferrule.

3. The system for controlling seizure episodes according to claim 1 wherein said service dog is trained to respond to changes in the patient's body chemistry associated with the onset of a seizure episode, and which the dog can distinguish by its sense of smell.

4. The system for controlling seizure episodes according to claim 1 wherein said service dog is trained to watch the patient's facial expression and to respond to changes in facial expression characteristic of the onset of a seizure episode.

5. A system for controlling seizure episodes in a patient who suffers from epilepsy and who is accompanied by a service dog trained to watch the patient and to respond to seizure episodes; the system comprising:
a cell phone with a dial pad that includes at least one button that can be set to cause the phone to dial an emergency contact number when the button is depressed;
a case into which the cell phone is fitted, the case being formed of a stiff material, the case possessing a protuberance inside the case that is positioned over said button, and which serves to depress said button and cause the cell phone to dial said emergency contact number in the event that the service dog grasps the cell phone and case in its mouth and begins to chew; and
a strap that is attached to one of the patient or the service dog and to the case of the cell phone, so that the latter is in position to be grasped by the service dog at the onset of a seizure episode for said patient.

6. The system of controlling seizure episodes according to claim 5 wherein said service dog is trained to identify, with its sense of smell, changes in the body chemistry of the patient that characterize the onset of a seizure episode.

7. The system of controlling seizure episodes according to claim 5, wherein said case has a leather outer shell and a stiff liner.

8. The system of controlling seizure episodes according to claim 5 wherein said protuberance is formed of a round-head rivet affixed in said case at the position of said button.
